(19) United States
(12) Patent Application Publication
(10) Pub. No.: US 2016/0116487 A1
(43) Pub. Date: Apr. 28, 2016

(54) IN VITRO DETECTION OF PRIONS IN BLOOD

(71) Applicants: Edward A. Hoover, Loveland, CO (US); Candace K. Mathiason, Windsor, CO (US)

(72) Inventors: Edward A. Hoover, Loveland, CO (US); Candace K. Mathiason, Windsor, CO (US)

(73) Assignee: Colorado State University Research Foundation, Fort Collins, CO (US)

(21) Appl. No.: 14/920,422

(22) Filed: Oct. 22, 2015

Related U.S. Application Data
(60) Provisional application No. 62/067,016, filed on Oct. 22, 2014.

(51) Int. Cl. G01N 33/68

(52) U.S. Cl. CPC G01N 33/6893; G01N 2800/2828

(57) ABSTRACT

A method of screening a blood sample for the presence of prions. The method includes the steps of collecting the blood sample in heparin, contacting the sample with a solution comprising recombinant prion protein (rPrP) and Thioflavin T (ThT), and measuring the resulting ThT fluorescence in the sample. The method can further include the step of freezing and thawing the sample prior to contacting the sample with a solution comprising recombinant prion protein (rPrP) and Thioflavin T (ThT). The method can also include the step of precipitating the prions in sodium phosphotungstic acid (NaPTA) prior to contacting the sample with a solution comprising recombinant prion protein (rPrP) and Thioflavin T (ThT).

### CROSS-REFERENCE TO RELATED APPLICATIONS

This application claims the benefit of U.S. Provisional Application No. 62/067,016, filed Oct. 22, 2014.

### STATEMENT OF GOVERNMENT INTEREST

This invention was made with government support under Grant Nos. R01 NS061902, N01 AI025491 and R01 AI093634 awarded by the National Institutes of Health. The government has certain rights in the invention.

### FIELD OF INVENTION

This invention relates to prion detection. More specifically, this invention relates to in vitro detection of prions in blood.

### BACKGROUND OF THE INVENTION

The hematogenous spread of prions in transmissible spongiform encephalopathy (TSE)-infected animals has long been hypothesized [Eklund, C. M., R. C. Kennedy, and W. J. Hadlow, Pathogenesis of scrapie virus infection in the mouse. J Infect Dis, 1967. 117(1): p. 15-22; Pattison, I. H. and G. C. Millson, Distribution of the scrapie agent in the tissues of experimentally inoculated goats. J Comp Pathol, 1962. 72: p. 233-44. Hadlow, W. J., et al., Course of experimental scrapie virus infection in the goat. J Infect Dis, 1974. 129(5): p. 559-67.], but evidence for the presence of prions in non-nervous/lymphoid tissues and blood was not available for several decades [Fraser, H. and A. G. Dickinson, Studies of the lymphoreticular system in the pathogenesis of scrapie: the role of spleen and thymus. J Comp Pathol, 1978. 88(4): p. 563-73; Race, R. E. and D. Ernst, Detection of proteinase K-resistant prion protein and infectivity in mouse spleen by 2 weeks after scrapie agent inoculation. J Gen Virol, 1992. 73: p. 3319-3323; Kitamoto, T., S. Mohri, and J. Tateishi, Organ Distribution of Proteinase-resistant Prion Protein in Humans and Mice with Creutzfeldt-Jakob Disease. J Gen Virol, 1989. 70: p. 3371-3379; Horiuchi, M., et al., A cellular form of prion protein (PrPc) exists in many non-neuronal tissues of sheep. J Gen Virol, 1995. 76: p. 2583-2587; Prowse, C. V. and A. Bailey, Validation of Prion Removal by Leucocyte-Depleting Filters: A Cautionary Tale. Vox Sang, 2000. 79: p. 248]. Later studies have provided unequivocal proof of efficient TSE blood-borne infectivity [Hunter, N., et al., Transmission of prion diseases by blood transfusion. J Gen Virol, 2002. 83: p. 2897-2905; Houston, F., et al., Prion diseases are efficiently transmitted by blood transfusion in sheep. Blood, 2008. 112(12): p. 4739-45; Andreoletti, O., et al., Highly efficient prion transmission by blood transfusion. PLoS Pathog, 2012. 8(6): p. e1002782; Lacroux, C., et al., Prionemia and leukocyte-platelet-associated infectivity in sheep transmissible spongiform encephalopathy models. J Virol, 2012. 86(4): p. 2056-66.]. The knowledge that prions traffic throughout the body in blood has important implications for both human and animal health.

Variant Creutzfeldt-Jakob disease (vCJD) emerged following the bovine spongiform encephalopathy (BSE) epidemic in the United Kingdom in the 1980s and 90s. Biochemical and strain typing analysis have provided evidence indicating that vCJD originated from human exposure to BSE contaminated material. To date, 225 cases of vCJD have been diagnosed worldwide [WHO, Variant Creutzfeldt-Jacob Disease. The World Health Organization, 2012. http://www.who.int/mediacentre/factsheets/fs180/en/], four of which have been transmitted by non-leucodepleted blood transfusion [Team, E., Fourth case of transfusion-associated vCJD infection in the United Kingdom. Euro Surveill, 2007. 12(1): p. E070118 4; Wroe, S. J., et al., Clinical presentation and pre-mortem diagnosis of variant Creutzfeldt-Jakob disease associated with blood transfusion: a case report. The Lancet, 2006. 368(9552): p. 2061-2067; Llewelyn, C. A., et al., Possible transmission of variant Creutzfeldt-Jakob disease by blood transfusion. The Lancet, 2004. 363(9407): p. 417-421; Peden, A. H., et al., Preclinical vCJD after blood transfusion in a PRNP codon 129 heterozygous patient. The Lancet, 2004. 364(9433): p. 527-529; Peden, A. H., et al., Advances in the development of a screening test for variant Creutzfeldt-Jakob disease. Expert Opin Med Diagn, 2008. 2(2): p. 207-19.]. While leucocyte reduction has been implemented to filter prions and prion carrying cells from blood products, current filtration methodologies are unable to remove 100% of TSE infectivity [Prowse, C. V. and A. Bailey, Validation of Prion Removal by Leucocyte-Depleting Filters: A Cautionary Tale. Vox Sang, 2000. 79: p. 248; Gregori, L., et al., Effectiveness of leucoreduction for removal of infectivity of transmissible spongiform encephalopathies from blood. The Lancet, 2004. 364(9433): p. 529-531. Lacroux, C., et al., Impact of leucocyte depletion and prion reduction filters on TSE blood borne transmission. PLoS One, 2012. 7(7): p. e42019.]. In addition, recent reports have revealed that 1/1,250 to 1/3,500 persons in the United Kingdom may be asymptomatic carriers of vCJD as a result of the BSE epidemic [Group, U.B.S.P.W. Creutzfeldt-Jakob Disease: Joint UKBTS/HPA Professional Advisory Committee Position Statement http://www.transfusionguidelines.org.uk/index.aspx?Publication=DL&Section=12&pag eid=794 2012; Collee, J. G., R. Bradley, and P. P. Liberski, Variant CJD (vCJD) and Bovine Spongiform Encephalopathy (BSE): 10 and 20 years on: part 2. Folia Neuropathol, 2006. 44(2): p. 102-110.]. Thus, concern exists that a secondary outbreak of vCJD may ensue involving blood-borne prion transmission [McCutcheon, S., et al., All Clinically-Relevant Blood Components Transmit Prion Disease following a Single Blood Transfusion: A Sheep Model of vCJD. PLOS ONE, 2011. 6(8): p. e23169.] originating from individuals unknowingly carrying a subclinical prion infection [Puopolo, M., et al., Transmission of sporadic Creutzfeldt-Jakob disease by blood transfusion: risk factor or possible biases. Transfusion, 2011. 51(7): p. 1556-66; Barrenetxea, G., Iatrogenic prion diseases in humans: an update. Eur J Obstet Gynecol Reprod Biol, 2012. 165(2): p. 165-9.]. Here we address the need for an in vitro assay with the ability to detect the prion disease-associated isoform of prion protein (PrP^{D}) present in whole blood.

Several animal TSEs, including chronic wasting disease (CWD) of deer and elk [Mathiason, C. K., et al., Infectious Prions in the Saliva and Blood of Deer with Chronic Wasting Disease. Science, 2006. 314: p. 133-136; Mathiason, C. K., et al., B cells and platelets harbor prion infectivity in the blood of deer infected with chronic wasting disease. J Virol, 2010. 84(10): p. 5097-107.] and hamster-adapted transmissible spongiform encephalopathy (TME) [Saa, P., J. Castilla, and C. Soto, Presymptomatic detection of prions in blood. Science, 2006. 313(5783): p. 92-4; Castilla, J., P. Saa, and C. Soto, Detection of prions in blood. Nat Med, 2005. 11(9): p. 982-5.] exhibit a hematogenous phase of infection, thus providing excellent TSE models for the development of an ante-mortem blood-borne PrP^{D} detection assay.

While traditional assays, such as Western blot and immunohistochemistry (IHC), are effective for detecting large quantities of prions present in nervous and lymphoid tissue, they do not have the ability to detect the minute quantities thought to be present in bodily fluids or peripheral tissues early in infection. Rodent bioassays have the necessary sensitivity and specificity to detect hematogenous prions, but they are not realistic as rapid and cost-effective diagnostic tools. In vitro prion detection was advanced with the advent of serial protein misfolding cyclic amplification (sPMCA) [Saa, P., J. Castilla, and C. Soto, Presymptomatic detection of prions in blood. Science, 2006. 313(5783): p. 92-4; Saa, P., J. Castilla, and C. Soto, Cyclic amplification of protein misfolding and aggregation. Methods Mol Biol, 2005. 299: p. 53-65.]. sPMCA has been optimized for the detection of prions in blood [Castilla, J., P. Saa, and C. Soto, Detection of prions in blood. Nat Med, 2005. 11(9): p. 982-5.] and requires less time than bioassay, but has been hampered by a lack of consistent sensitivity and a dependence on protease digestion prior to immunoassay readout. In contrast, the real-time quaking-induced conversion (RT-QuIC) assay [Atarashi, R., et al., Ultrasensitive detection of scrapie prion protein using seeded conversion of recombinant prion protein. Nat Methods, 2007. 4(8): p. 645-50; Orrú, C. D., et al., Prion Disease Blood Test Using Immunoprecipitation and Improved Quaking-Induced Conversion. mBio, 2011. 2(3): p. e00078-11; Wilham, J. M., et al., Rapid end-point quantitation of prion seeding activity with sensitivity comparable to bioassays. PLoS Pathog, 2010. 6(12): p. e1001217.] relies upon the seeded conversion of recombinant prion protein (rPrP) to PrP^{D} and subsequent binding of thioflavin T (ThT) to the resulting amyloid isoforms [Krebs, M. R., E. H. Bromley, and A. M. Donald, The binding of thioflavin-T to amyloid fibrils: localisation and implications. J Struct Biol, 2005. 149(1): p. 30-7.], thus offering enhanced ante-mortem prion detection and real-time fluorescence readout [Wilham, J. M., et al., Rapid end-point quantitation of prion seeding activity with sensitivity comparable to bioassays. PLoS Pathog, 2010. 6(12): p. e1001217.].

The present invention demonstrates that adaptations of the RT-QuIC provide a fast, sensitive and consistent assay for the detection of blood-borne prions.

### SUMMARY OF THE INVENTION

Blood-borne transmission of infectious prions during the asymptomatic or pre-clinical stage of disease occurs for both human and animal transmissible spongiform encephalopathies (TSEs). The geographical distribution of the cervid TSE, chronic wasting disease (CWD), continues to spread across North America and the prospective number of individuals harboring an asymptomatic infection of human variant Creutzfeldt-Jakob Disease (vCJD) in the United Kingdom has been projected to be ˜1 in 3000 residents. Thus, it is important to monitor cervid and human blood products to ensure herd health and human safety. Current methods for detecting blood-associated prions rely primarily upon bioassay in laboratory animals. While bioassay provides high sensitivity and specificity, it requires many months, animals, and costs. Modifications of the real time quaking-induced conversion (RT-QuIC) assay enables detection of blood-borne prions in whole blood from prion-infected preclinical white-tailed deer, muntjac deer, and Syrian hamsters, attaining sensitivity of >90% while maintaining 100% specificity. Our results indicate that RT-QuIC methodology as modified can provide consistent and reliable detection of blood-borne prions in pre-clinical and symptomatic stages of two animal TSEs, offering promise for prionemia detection in other species, including humans.

The long-standing but heretofore unfulfilled need for is now met by a new, useful, and nonobvious invention. In a first aspect the present invention provides a method of screening a blood sample for the presence of prions. The method includes the steps of collecting the blood sample in heparin, contacting the sample with a solution comprising recombinant prion protein (rPrP) and Thioflavin T (ThT), and measuring the resulting ThT fluorescence in the sample. In an advantageous embodiment about 200 units/ml of heparin are added to the sample. The method can further include the step of freezing and thawing the sample prior to contacting the sample with a solution comprising recombinant prion protein (rPrP) and Thioflavin T (ThT). In performing the freezing and thawing step aliquots of whole blood can be frozen at about −80° C. for about 30 minutes and subsequently thawed at about 22° C. for about 60 minutes. In an advantageous embodiment the samples can be subjected to at least four (4) freeze-thaw cycles. In further advantageous embodiments the sample can be homogenized after the freezing and thawing steps. The method can further include the step of precipitating the prions in sodium phosphotungstic acid (NaPTA) prior to contacting the sample with a solution comprising recombinant prion protein (rPrP) and Thioflavin T (ThT). In an advantageous embodiment the samples can be subjected to at least one (1) freeze-thaw cycle prior to the precipitation step.

### BRIEF DESCRIPTION OF THE DRAWINGS

For a fuller understanding of the invention, reference should be made to the following detailed description, taken in connection with the accompanying drawings, in which:

FIG. 1 is a series of three graphs (1A-1C) showing RT-QuIC analysis of whole blood collected in various anticoagulants. Blood was collected from a CWD-infected and CWD-naïve white-tailed deer and preserved in one of three anticoagulants: CPDA, EDTA or heparin. Serial blood sample dilutions (neat to 10⁻⁶) were assayed by RT-QuIC for 60 hours and ThT fluorescence level above threshold determined positivity. Detection of PrP^{C} converting activity for each replicate is shown for blood collected in CPDA (A), EDTA (B) and heparin (C).

FIG. 2 is a pair of graphs (2A-2B) showing RT-QuIC analysis of fresh versus frozen whole blood. Blood was collected from a CWD-infected and CWD-naïve white-tailed deer and aliquots were analyzed immediately (fresh) or frozen (−80 C). Serial blood sample dilutions (neat to 10⁻⁶) were assayed by RT-QuIC in duplicate for 60 hours and ThT fluorescence level above threshold determined positivity. Detection of PrP_{C} converting activity for each replicate is shown for blood analyzed fresh (A) and frozen (B).

FIG. 3 is a series of six graphs (3A-3F) showing RT-QuIC analysis of samples before and after treatment with NAPTA. Samples were untreated or precipitated using NAPTA, serially diluted (neat to 10⁻⁶), and assayed by RT-QuIC in triplicate for 60 hours. ThT fluorescence level above threshold determined positivity, each replicate is present. (A) Limited detection is seen in untreated blood samples. (B-D) Improved detection of PrP^{C} converting activity is seen in blood samples precipitated with NAPTA from CWD-infected white-tailed deer (B and C). No PrP^{C} converting activity was seen in samples precipitated with NAPTA from a CWD-naïve white-tailed deer (D).

FIG. 4 is a graph showing RT-QuIC comparison of brain and blood samples. Ten percent (10%) brain homogenates were serially diluted (10⁻⁵ to 10⁻⁸) and assayed by RT-QuIC for 60 hours. Blood samples were diluted to 10⁻² and run in triplicate for 60 hours with ThT fluorescence level above threshold determining positivity. (A) CWD-infected blood diluted 10⁻² is seen to have similar levels of PrP^{C} converting activity as CWD-positive brain diluted 10⁻⁶ and 10⁻⁷. UN=Uninfected; INF=Infected.

FIG. 5 is a series of six graphs (5A-5F) showing RT-QuIC analysis of cervid whole blood samples. Blood samples were diluted to 10⁻² and 8 replicates were analyzed over 2 runs of 60 hours, and positivity was determined by ThT fluorescence level above threshold. PrP^{C} converting activity is demonstrated in 22 CWD infected cervid blood samples, and is absent in all CWD-naïve samples (A-F). Each line is the average of four replicates for a specific animal. UN=Uninfected; INF=Infected.

FIG. 6 is a series of four graphs (A-D) showing RT-QuIC analysis of hamster whole blood samples. Blood samples were diluted to 10⁻² and 8 replicates were analyzed over 2 experiments of 60 hours, and positivity was determined by ThT fluorescence level above threshold. PrP^{C} converting activity is demonstrated in 21 TME-infected blood samples, and is absent in all TME-naïve samples (A-D). Each line is the average of four replicates for a specific animal. UN=Uninfected; INF=Infected.

FIG. 7 is a series of six images (7A-7F) showing PrP^{D} detection in hamster, white-tailed deer and muntjac by IHC. PrP^{D} immunoreactivity in a spinal cord tissue section from a hamster 16 weeks after extranasal inoculation with HY-TME (A) detected with antibody 3F4 and ABC solution. PrPD immunoreactivity in the brainstem of CWD-infected white-tailed deer (C) and muntjac (E) detected with antibody BAR224 and AEC substrate. No immunoreactivity was seen in the corresponding tissues of mock-inoculated controls (B, D and E). The boxed areas are enlarged 10× in the insets. Scale bar=200 μm.

FIG. 8 is a series of two graphs (8A-8B) showing RT-QuIC analysis of serially diluted cervid and hamster brain samples. Brain samples were serially diluted 10⁻³ to 10⁻⁶ or 10⁻³ to 10⁻¹⁰ for cervids (A) and hamsters (B), respectively, and analyzed in RT-QuIC for 60 hours. A ThT fluorescence level above threshold determined positivity. Both cervid and hamster brains from positively inoculated animals demonstrated positivity in all dilutions, while all brain dilutions from naïve animals remained negative.

### DETAILED DESCRIPTION OF THE PREFERRED EMBODIMENT

Prions are present in blood and have been unknowingly transmitted to recipients of blood donations from individuals in the pre-symptomatic phase of infection. Currently there is no test to screen blood for the presence of prions. This coupled with the requirement for human blood products in the UK to undergo intense processing for prion removal (<100% effective) has revealed a need for a diagnostic test with the ability to quickly and consistently detect low levels of prions in blood. Lack of efficient diagnostic assays also contributes to an inability to monitor animal populations for disease. We demonstrate herein that prions can be reliably detected in whole blood collected from various animal species infected with different strains of prions prior to the development of symptoms of disease. The application of this assay provides a means to survey blood from different populations and investigate early events associated with prion infection.

The development of a reliable in vitro blood-borne TSE-detection assay would have significant advantages for both human and animal populations and may provide a stepping-stone for the development of diagnostic assays for other protein misfolding diseases. To date, various in vitro assays have been developed with the goal of detecting prions present in blood [Orru, C. D., et al., New generation QuIC assays for prion seeding activity. Prion, 2012. 6(2): p. 147-52.]. Of particular note are sPMCA [Saa, P., J. Castilla, and C. Soto, Presymptomatic detection of prions in blood. Science, 2006. 313(5783): p. 92-4; Castilla, J., P. Saa, and C. Soto, Detection of prions in blood. Nat Med, 2005. 11(9): p. 982-5.], a ligand based assay developed to detect hematogenous prions [Terry, L. A., et al., Detection of PrPsc in blood from sheep infected with the scrapie and bovine spongiform encephalopathy agents. J Virol, 2009. 83(23): p. 12552-8.], and immunoprecipitation enhanced RT-QuIC [Orrú, C. D., et al., Prion Disease Blood Test Using Immunoprecipitation and Improved Quaking-Induced Conversion. mBio, 2011. 2(3): p. e00078-11]. However, demonstrating satisfactory sensitivity and specificity with these assays has been a challenge.

We demonstrate herein the in vitro detection of prionemia in CWD and TME-infected hosts during both pre-clinical and clinical phases of disease, establishing the merits of RT-QuIC as an effective antemortem diagnostic tool. Early detection and screening applications will provide a means to detect asymptomatic carriers of TSE disease in the human donor blood and tissue pools, thus indicating which samples should be eliminated. The ability to detect infected blood will aid in establishing monitoring parameters for TSE intervention/therapeutic strategies and provide domestic and wildlife herd management professionals with a live test for TSE surveillance.

### Example 1

### RT-QuIC Analysis of Whole Blood Collected in Various Anticoagulants

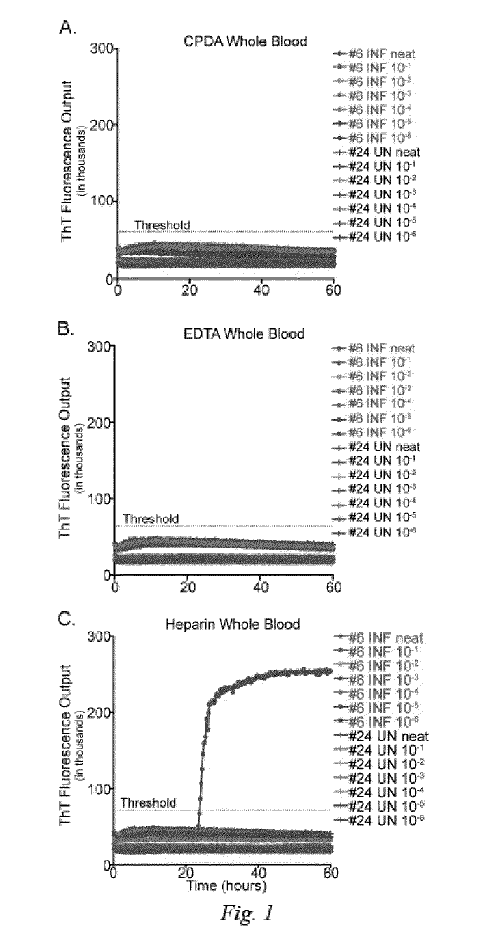

To determine the influence of common blood preservation reagents in in vitro PrP^{D} detection assays, we compared the ability of RT-QuIC to amplify CWD prions in cervid whole blood preserved in CPDA, EDTA or heparin. Samples were run in serial dilutions (10⁰-10⁻⁶) in the RT QuIC assay to determine the optimal dilution for PrP^{D} detection. While RT-QuIC PrP^{C} converting activity was observed in heparinized blood from CWD-infected deer (1/2 replicates in one dilution; 10⁻⁵), PrP^{C} converting activity was not detected in CPDA or EDTA preserved blood from the same animal or any blood collected from sham-inoculated deer (FIG. 1). All subsequent RT-QuIC analysis was conducted on whole blood harvested in heparin.

Precedence for hematogenous spread of prions via transfusion has been well established with various TSEs, including scrapie [Andreoletti, O., et al., Highly efficient prion transmission by blood transfusion. PLoS Pathog, 2012. 8(6): p. e1002782.], CWD [Mathiason, C. K., et al., Infectious Prions in the Saliva and Blood of Deer with Chronic Wasting Disease. Science, 2006. 314: p. 133-136; Mathiason, C. K., et al., Infectious Prions in Pre-Clinical Deer and Transmission of Chronic Wasting Disease Solely by Environmental Exposure. PLOS ONE, 2009. 4(6): p. e5916.], BSE [Houston, F., et al., Prion diseases are efficiently transmitted by blood transfusion in sheep. Blood, 2008. 112(12): p. 4739-45.] and vCJD [Team, E., Fourth case of transfusion-associated vCJD infection in the United Kingdom. Euro Surveill, 2007. 12(1): p. E070118 4; Wroe, S. J., et al., Clinical presentation and pre-mortem diagnosis of variant Creutzfeldt-Jakob disease associated with blood transfusion: a case report. The Lancet, 2006. 368(9552): p. 2061-2067; Llewelyn, C. A., et al., Possible transmission of variant Creutzfeldt-Jakob disease by blood transfusion. The Lancet, 2004. 363(9407): p. 417-421; Peden, A. H., et al., Preclinical vCJD after blood transfusion in a PRNP codon 129 heterozygous patient. The Lancet, 2004. 364(9433): p. 527-529; McCutcheon, S., et al., All Clinically-Relevant Blood Components Transmit Prion Disease following a Single Blood Transfusion: A Sheep Model of vCJD. PLOS ONE, 2011. 6(8): p. e23169.]. To date, few in vitro assays are capable of detecting prions present in the blood of infected individuals, and those that do can suffer from decreased sensitivity due to the presence of assay inhibitors [Castilla, J., P. Saa, and C. Soto, Detection of prions in blood. Nat Med, 2005. 11(9): p. 982-5; Terry, L. A., et al., Detection of PrPsc in blood from sheep infected with the scrapie and bovine spongiform encephalopathy agents. J Virol, 2009. 83(23): p. 12552-8; Bannach, O., et al., Detection of prion protein particles in blood plasma of scrapie infected sheep. PLoS One, 2012. 7(5): p. e36620; Edgeworth, J. A., et al., Detection of prion infection in variant Creutzfeldt-Jakob disease: a blood-based assay. The Lancet, 2011. 377: p. 487-493.].

To assess whether anticoagulants affect PrP^{D} detection, we analyzed whole blood collected in CPDA, EDTA and heparin. It has been demonstrated in previous work [Glier, H. and K. Holada, Blood storage affects the detection of cellular prion protein on peripheral blood leukocytes and circulating dendritic cells in part by promoting platelet satellitism. J Immunol Methods, 2012. 380(1-2): p. 65-72.] that anticoagulant storage can affect the presentation of cellular PrP. Here, we have shown that whole blood collected in heparin, but not in CPDA and EDTA, elicited efficient in vitro RT-QuIC prion conversion. It has been shown that polyanions enhance the amplification of prions in in vitro conversion assays, suggesting that they may contribute to conversion efficiency [Deleault, N. R., et al., Protease-resistant prion protein amplification reconstituted with partially purified substrates and synthetic polyanions. J Biol Chem, 2005. 280(29): p. 26873-9; Wong, C. n., et al., Sulfated glycans and elevated temperature stimulate PrPSc-dependent cell-free formation of protease-resistant prion protein. The EMBO Journal, 2001. 20(3): p. 377-386.]. Heparin, a polyanion, has previously been shown to enhance in vitro detection of PrP^{D} [Yokoyama, T., et al., Heparin enhances the cell-protein misfolding cyclic amplification efficiency of variant Creutzfeldt-Jakob disease. Neurosci Lett, 2011. 498(2): p. 119-23.] and is thought to serve as a potential cofactor in prion propagation in vivo by acting as a scaffolding molecule or catalyst due to its highly negative charged-glycosaminoglycan nature [Deleault, N. R., et al., Formation of native prions from minimal components in vitro. Proc Natl Acad Sci USA, 2007. 104(23): p. 9741-6.]. EDTA and CPDA owe their anticoagulant nature to their ability to chelate calcium in blood. Chelating agents are widely used for scavenging metal ions [Dominguez, K. and W. S. Ward, A novel nuclease activity that is activated by Ca(2+) chelated to EGTA. Syst Biol Reprod Med, 2009. 55(5-6): p. 193-9.] which may contribute to the absence of PrP^{C} conversion observed in blood collected in the two anticoagulants. Further research is needed to determine the role anticoagulants play in inhibiting/facilitating RT-QuIC. We have demonstrated that preserving whole blood samples in heparin may facilitate in vitro prion detection.

### Example 2

### RT-QuIC Analysis of Fresh Versus Frozen Whole Blood

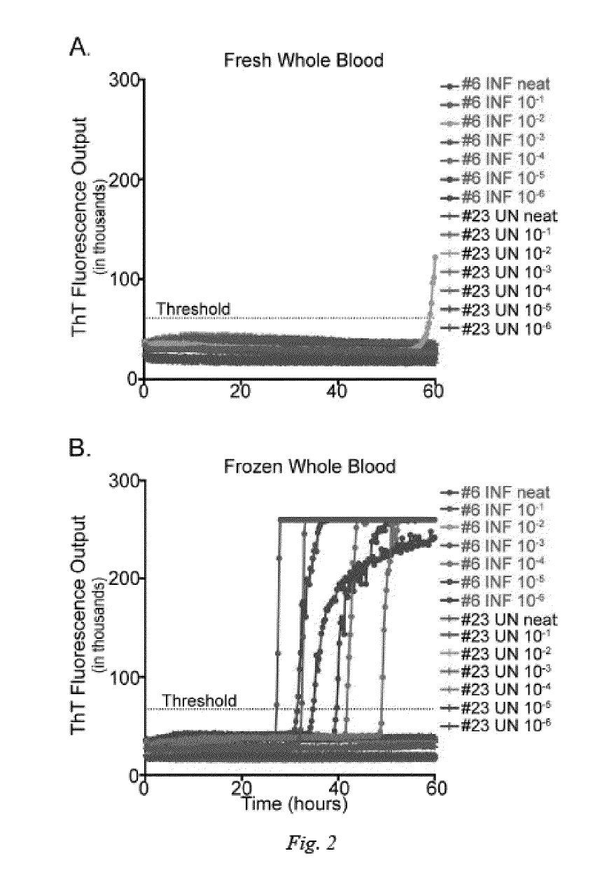

In order to determine if historical blood samples were adequately preserved to initiate PrP^{C} converting activity in RT-QuIC, whole blood was collected from contemporary naïve and CWD112 infected white-tailed deer and compared as fresh versus frozen samples. Samples were processed in various dilutions ranging from undiluted to 10⁻⁶ to determine the optimal dilution for PrP^{D} detection using frozen whole blood in the RT-QuIC assay. While PrP^{C} converting activity was detected in fresh whole blood, blood that had been processed through the freeze-thaw procedure yielded higher and more consistent detection of prion converting activity (2/2 replicates in each of four dilutions) (FIG. 2). PrP^{C} converting activity was not observed in wells containing only substrate or naïve cervid blood. All subsequent RT-QuIC analysis included heparinized whole blood that had undergone four freeze-thaw cycles.

To assess the feasibility of using historical frozen samples for future analysis of blood-borne prions, we evaluated the effects of freezing blood prior to RT-QuIC. We have demonstrated that the freeze-thaw cycle enhances RT-QuIC blood-borne prion detection sensitivity, facilitating in vitro prion detection at earlier time points with a more robust amplification than samples that did not undergo the freeze-thaw process. There is compelling evidence for the accumulation of aggregated misfolded prion isoforms in the cytoplasm of infected cells [Hofmann, J. P., et al., Cell-to-cell propagation of infectious cytosolic protein aggregates. Proc Natl Acad Sci USA, 2013. 110(15): p. 5951-6; Aguzzi, A. and L. Rajendran, The transcellular spread of cytosolic amyloids, prions, and prionoids. Neuron, 2009. 64(6): p. 783-90.] and it is hypothesized that these aggregates are released from the cell as lysis occurs. Thermal shock on whole blood samples damages the cell membrane and initiates hemolysis [Lovelock, J. E., Physical instability and thermal shock in red cells. Nature, 1954. 173(4406): p. 659-61; Lovelock, J. E., Haemolysis by thermal shock. Br J Haematol, 1955. 1(1): p. 117-29.], which is thought to release intracellular components. Cell lysis of blood collected from TSE-infected animals, associated with repeated freeze-thaw cycles may liberate sufficient prions to enhance in vitro nucleation and thus the detection of PrP^{C} converting activity.

### Example 3

### Effects of Sodium Phosphotungstic Acid Precipitation (NaPTA) on RT-QuIC PrP^{D} Detection

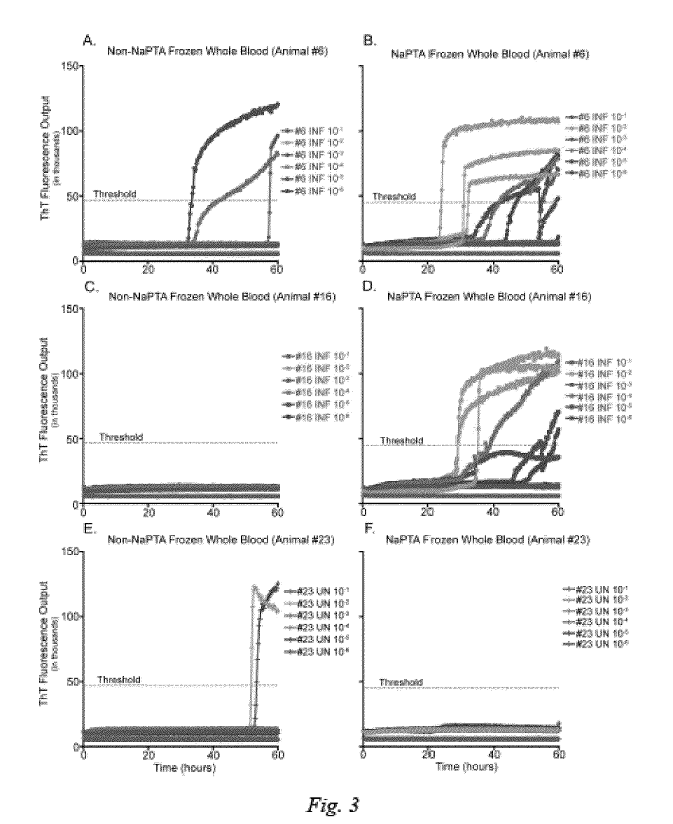

NaPTA precipitation was applied to heparinized whole blood that had undergone freeze-thaw cell lysis in an attempt to increase both the sensitivity and specificity of the RT-QuIC assay. With the improved sensitivity and specificity provided by NaPTA pretreatment we were able to demonstrate reliable RT-QuIC results at a 10⁻² dilution of CWD-infected whole blood, while NaPTA treated whole blood from a naïve individual remained conversion free (FIG. 3).

Thus, all of the remaining RT-QuIC analyses of TSE prion converting activity in historical and contemporary samples were conducted with heparinized and freeze-thawed NaPTA-treated whole blood.

It has been suggested that there are components present in bodily fluids that interfere with or inhibit prion conversion and thus in vitro detection of the aberrant form of the prion protein [Barria, M. A., D. Gonzalez-Romero, and C. Soto, Cyclic amplification of prion protein misfolding. Methods Mol Biol, 2012. 849: p. 199-212; Chen, B., et al., Estimating prion concentration in fluids and tissues by quantitative PMCA. Nature Methods, 2010. 7(7): p. 519-521.]. Various groups have attempted to solve this problem using different concentration methods. Using immunoprecipitation coupled with RT-QuIC, Orrú et al. [Orrú, C. D., et al., Prion Disease Blood Test Using Immunoprecipitation and Improved Quaking-Induced Conversion. mBio, 2011. 2(3): p. e00078-11.] were able to establish in vitro detection of PrP^{C} converting activity in plasma and serum samples from scrapie-infected hamsters. Morales et al. [Morales, R., et al., Reduction of prion infectivity in packed red blood cells. Biochem Biophys Res Commun, 2008. 377(2): p. 373-8.] demonstrated that the use of varying concentrations of sarkosyl could concentrate PrP^{D} present in tissue and fluid samples. Wadsworth and colleagues [Wadsworth, J. D. F., et al., Tissue distribution of protease resistant prion protein in variant Creutzfeldt-Jakob disease using a highly sensitive immunoblotting assay. The Lancet, 2001. 358(9277): p. 171-180; D'Castro, L., et al., Isolation of Proteinase K-Sensitive Prions Using Pronase E and Phosphotungstic Acid. PLOS ONE, 2010. 5(12): p. e15679.] have shown that sarkosyl coupled with the use of sodium phosphotungstic acid enhances the isolation of both PrP^{C} and PrP^{D} from bodily fluids. Using NaPTA precipitation we were able to concentrate hematogenous prions to a more detectable level and/or remove assay inhibitors, augmenting our ability to directly detect prions in whole blood.

With the application of an anticoagulant that facilitates prion conversion in vitro, the freeze-thaw cell lysis and NaPTA precipitation, we have optimized the RT-QuIC assay for efficient detection of PrP^{D} in whole blood samples, thus we are calling our new protocol whole blood optimized (WBO) RT-QuIC. NaPTA precipitation increased the number of positive replicates and decreased the assay time required to initiate PrP^{C} conversion/detection in whole blood harvested from TSE-infected animals while limiting false positive PrP^{C} converting activity in samples from uninfected animals.

### Example 4

### RT-QuIC Comparison of CWD-Positive Brain Versus NaPTA Concentrated Whole Blood

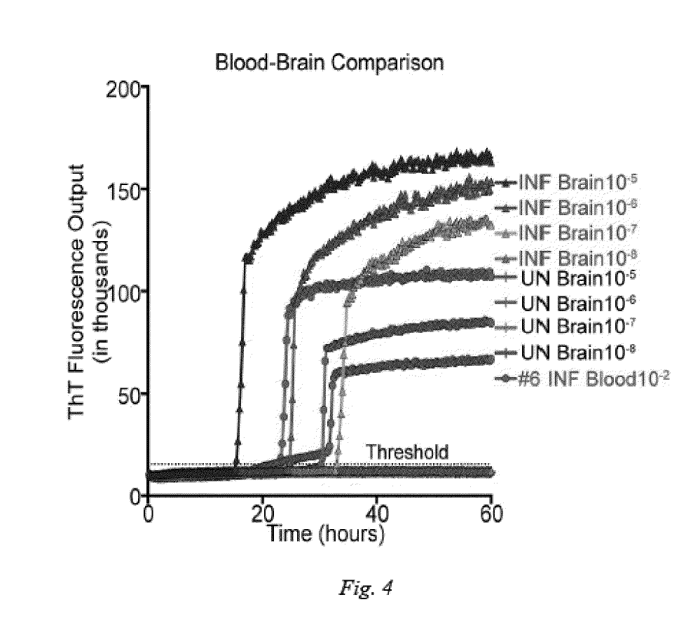

To evaluate the levels of PrP^{D} present in NaPTA concentrated whole blood samples, PrP^{C} converting activity was compared to that detected in serial dilutions of CWD-positive white tailed deer brain (FIG. 4). NaPTA treated whole blood (10 ml starting volume of whole blood) diluted to 10⁻² demonstrated PrP^{D} levels approximately equivalent to that measured in 10⁻⁶-10⁻⁷ dilution of CWD-positive brain. Equivalence was determined by comparison of the time to positivity for whole blood and brain samples.

Many groups have developed quantitative in vitro methodologies to analyze the levels of PrP^{D} present in various tissues and bodily fluid samples. Murayama et al. [Murayama, Y., et al., Urinary excretion and blood level of prions in scrapie-infected hamsters. J Gen Virol, 2007. 88(Pt 10): p. 2890-8.] used PMCA to establish a direct comparison of PrP^{D} levels in buffy coat and plasma to PrPD levels seen in serial dilutions of TSE-infected brain by analyzing which round of PMCA samples began demonstrating positivity. Other laboratories [Castilla, J., P. Saa, and C. Soto, Detection of prions in blood. Nat Med, 2005. 11(9): p. 982-5; Chen, B., et al., Estimating prion concentration in fluids and tissues by quantitative PMCA. Nature Methods, 2010. 7(7): p. 519-521; Gonzalez-Romero, D., et al., Detection of infectious prions in urine. FEBS Lett, 2008. 582(21-22): p. 3161-6.] have reported quantitative and semi-quantitative methods of PMCA to determine the levels of PrP^{D} in blood and urine by comparing to the amount of amplifiable PrP^{D} present in TSE-infected brain. Castilla et al. [Castilla, J., P. Saa, and C. Soto, Detection of prions in blood. Nat Med, 2005. 11(9): p. 982-5.] were able to demonstrate that PMCA amplifiable prions in buffy coat collected from 1 ml of scrapie-adapted hamster blood contained roughly 0.1-1 pg of PrP^{D} molecules. Our RT-QuIC results indicate that 2 μl of a 10⁻² dilution (0.5 ml of whole blood NaPTA precipitated 10-fold, further diluted to 10⁻²) contained PrP^{D} levels equivalent to those seen in 0.2 ng-2 ng of CWD-positive brain.

### Example 5

### Detection of Prion Converting Activity in CWD-Infected Cervid Whole Blood

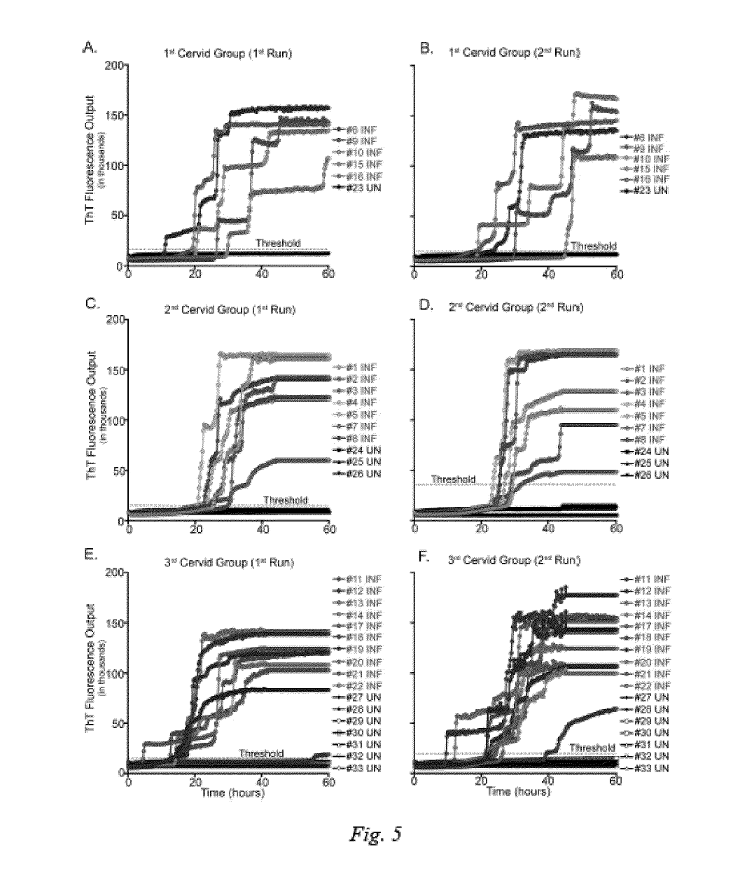

Twenty-two of 22 clinical and sub-clinical CWD-infected cervids (16 white-tailed deer and 6 muntjac deer) and 0/11 naive cervids (5 white-tailed deer and 6 muntjac deer) exhibited RT143 QuIC PrP^{C} converting activity in 7/8 or 8/8 replicates within 60 hours (FIG. 5, Table 1). Sample replicates were averaged on each plate and a positive threshold was set at five times the standard deviation of the negative control average.

TABLE 1
Cervid blood donor inoculation, clinical status, and assay results

| | | | | | | Posi- |
|---|---|---|---|---|---|---|
| | | | | Western | | tive |
| | | Route of | | Blot | | QuIC |
| | | Inocu- | Disease | Status | IHC | Repli- |
| Animal # | Inoculum | lation | Status | (Obex) | Status | cates |
| 1 (WTD) | 2 ml 5% CWD + | Aerosol | Clinical | + | +^{B} | 8/8 |
| | brain homogenate | | | | | |
| 2 (WTD) | 2 ml 5% CWD + | Aerosol | Clinical | + | +^{B} | 8/8 |
| | brain homogenate | | | | | |
| 3 (WTD) | 2 ml 5% CWD + | Aerosol | Clinical | + | +^{B} | 7/8 |
| | brain homogenate | | | | | |
| 4 (WTD) | 2 ml 5% CWD + | Aerosol | Clinical | + | +^{B} | 8/8 |
| | brain homogenate | | | | | |
| 5 (WTD) | 2 ml 5% CWD + | Aerosol | Sub- | + | +^{B} | 8/8 |
| | brain homogenate | | clinical | | | |
| 6 (WTD) | 2 ml 5% CWD + | Aerosol | Clinical | + | +^{B} | 8/8 |
| | brain homogenate | | | | | |
| 7 (WTD) | 1.0 g 10% CWD + | PO | Sub- | ND | +^{B} | 7/8 |
| | brain homogenate | | clinical | | | |
| 8 (WTD) | 1.0 g 10% CWD + | PO | Sub- | + | +^{B} | 8/8 |
| | brain homogenate | | clinical | | | |
| 9 (WTD) | 1.0 g 10% CWD + | PO | Sub- | − | −^{B} | 7/8 |
| | brain homogenate | | clinical | | | |
| 10 (WTD) | 1.0 g 10% CWD + | PO | Sub- | + | +^{B} | 8/8 |
| | brain homogenate | | clinical | | | |
| 11 (WTD) | 1.0 g 10% CWD + | PO | Sub- | + | +^{B} | 8/8 |
| | brain homogenate | | clinical | | | |
| 12 (WTD) | 1.0 g 10% CWD + | PO | Clinical | + | +^{B} | 7/8 |
| | brain homogenate | | | | | |
| 13 (WTD) | 1.0 g 10% CWD + | PO | Clinical | + | +^{B} | 8/8 |
| | brain homogenate | | | | | |
| 14 (WTD) | 1.0 g 10% CWD + | PO | Sub- | + | +^{B} | 7/8 |
| | brain homogenate | | clinical | | | |
| 15 (WTD) | 2.0 g 10% CWD + | IC | Clinical | + | +^{B,O} | 7/8 |
| | brain homogenate | | | | | |
| 16 (WTD) | 250 ml 5% CWD + | IV | Clinical | + | +^{B,O} | 8/8 |
| | whole blood | | | | | |
| 17 (MJ) | 1.0 g 10% CWD + | PO/SQ | Sub- | − | +^{O} | 8/8 |
| | brain homogenate | | clinical* | | | |
| 18 (MJ) | 1.0 g 10% CWD + | PO/SQ | Sub- | − | +^{O} | 6/8 |
| | brain homogenate | | clinical* | | | |
| 19 (MJ) | 1.0 g 10% CWD + | PO/SQ | Clinical | + | +^{B,O} | 8/8 |
| | brain homogenate | | | | | |
| 20 (MJ) | 1.0 g 10% CWD + | PO/SQ | Clinical | + | +^{B,O} | 8/8 |
| | brain homogenate | | | | | |
| 21 (MJ) | 1.0 g 10% CWD + | PO/SQ | Clinical | + | +^{B,O} | 8/8 |
| | brain homogenate | | | | | |
| 22 (MJ) | 1.0 g 10% CWD + | PO/SQ | Clinical | + | +^{B,O} | 4/8 |
| | brain homogenate | | | | | |
| 23 (WTD) | 2 ml sham | Aerosol | NA | − | −^{B} | 0/8 |
| | homogenate | | | | | |
| 24 (WTD) | 2 ml sham | Aerosol | NA | − | −^{B} | 0/8 |
| | homogenate | | | | | |
| 25 (WTD) | 2 ml sham | Aerosol | NA | − | −^{B} | 0/8 |
| | homogenate | | | | | |
| 26 (WTD) | CWD— | PO | NA | − | −^{B,O} | 0/8 |
| | urine/feces | | | | | |
| 27 (WTD) | CWD— | PO | NA | − | −^{B,O} | 0/8 |
| | urine/feces | | | | | |
| 28 (MJ) | 1.0 g sham | PO/SQ | NA | − | _^{O} | 0/8 |
| | homogenate | | | | | |
| 29 (MJ) | 1.0 g sham | PO/SQ | NA | − | −^{O} | 0/8 |
| | homogenate | | | | | |
| 30 (MJ) | 1.0 g sham | PO/SQ | NA | − | −^{O} | 0/8 |
| | homogenate | | | | | |
| 31 (MJ) | Uninoculated | NA | NA | − | −^{O} | 0/8 |
| 32 (MJ) | Uninoculated | NA | NA | − | −^{O} | 0/8 |
| 33 (MJ) | Uninoculated | NA | NA | − | −^{O} | 0/8 |

WTD = White-tailed deer
MJ = Muntjac deer
ND = Not Done
NA = Not Available
− = PrP^{D} was not detected in the sample
+ = PrP^{D} was detected in the sample
^{B}= Biopsy of tonsil and recto-anal mucosa associated lymphoid tissue
^{O}= Obex
*= Less than halfway to clinical disease

Wilham et al. [Wilham, J. M., et al., Rapid end-point quantitation of prion seeding activity with sensitivity comparable to bioassays. PLoS Pathog, 2010. 6(12): p. e1001217.] demonstrated that the RT-QuIC assay has the ability to detect prions in tissue samples with similar sensitivity as bioassay (˜1 lethal dose), rendering it appropriate for the detection of PrP^{D} in bodily fluids such as blood and saliva. RT-QuIC assay efficacy for CWD-infected whole blood was evaluated following pretreatment to augment the release of prions from carrier cells and minimize inhibitory factors (freeze-thaw/NaPTA). We have demonstrated that our optimized RT-QuIC assay is sufficiently sensitive to detect PrP^{C} converting activity in whole blood harvested from pre-clinical and clinical IHC/Western blot-confirmed CWD-infected animals. Furthermore, our optimized RT-QuIC assay has demonstrated the ability to detect PrP^{C} converting activity in CWD-inoculated animals prior to the mid point between inoculation and clinical disease.

Using PMCA for the detection of PrP^{D} in the blood of scrapie-infected hamsters, Saa et al. [Saa, P., J. Castilla, and C. Soto, Presymptomatic detection of prions in blood. Science, 2006. 313(5783): p. 92-4.] reported sensitivity levels of 80% for clinical animals, and up to 60% for pre-clinical animals. Orrú et al. demonstrated even greater sensitivity for PrP^{D} in blood plasma of scrapie-infected hamsters using immunoprecipitation coupled with RT-QuIC [Orrú, C. D., et al., Prion Disease Blood Test Using Immunoprecipitation and Improved Quaking-Induced Conversion. mBio, 2011. 2(3): p. e00078-11.]. Utilizing our optimized RT QuIC assay for cervid whole blood, we have exhibited sensitivity levels of 93.8% for clinical animals, and 92.2% for sub-clinical animals while maintaining 100% specificity for both groups. These results reveal the potential of RT-QuIC as a reliable in vitro assay for blood-borne prion detection.

### Example 6

### Detection of Prion Converting Activity in TME-Infected Hamster Whole Blood

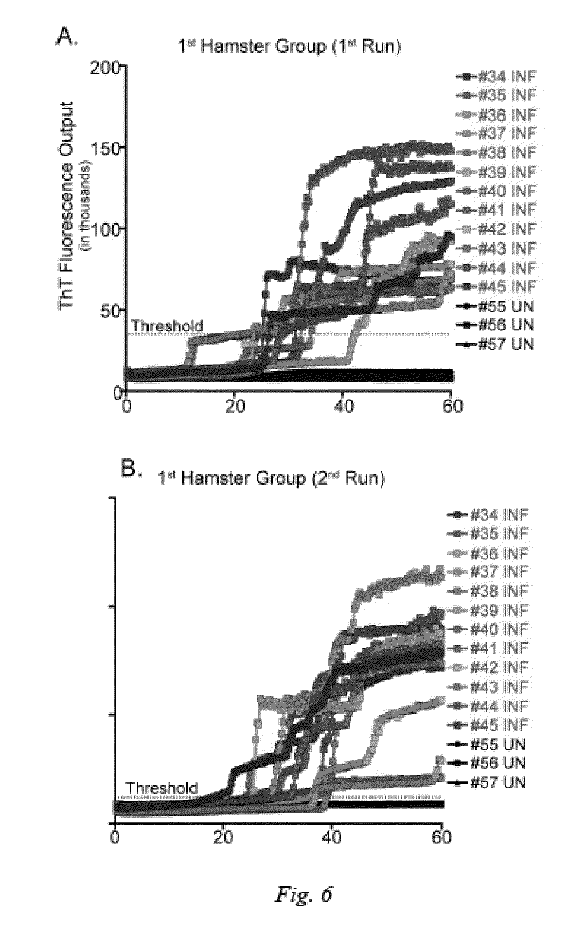

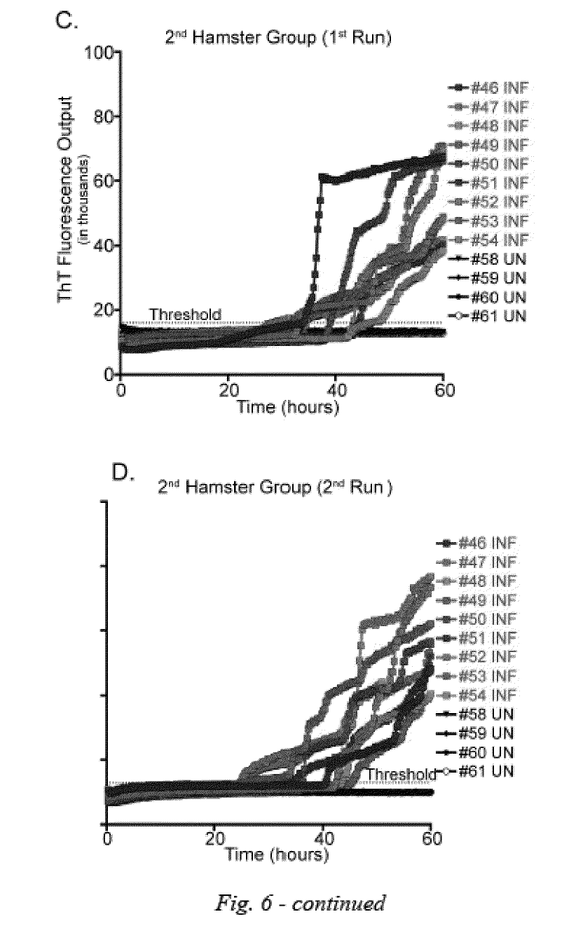

The hyper strain of transmissible mink encephalopathy (HY TME) was chosen for the RT-QuIC assay to determine the assays ability for PrPD detection in various species and strains of TSEs. All HY TME-infected hamsters (n=21) ranging from 6 to 20 weeks post infection exhibited RT151 QuIC PrPC converting activity in 5/8-8/8 replicates within 60 hours, while all (n=7) of the age matched controls failed to seed RT-QuIC (FIG. 6, Table 2). As above, sample replicates were averaged on each plate and a positive threshold was set at five times the standard deviation of the negative control average.

TABLE 2
Hamster blood donor inoculation, clinical status, and assay results

| | | | | | | Posi- |
|---|---|---|---|---|---|---|
| | | | | Sample | | tive |
| | | | | Collec- | | QuIC |
| Animal | | Route of | Disease | tion | IHC | Repli- |
| # | Inoculum | Inoculation | Status | Date | Status | cates |
| 34 | 10 μl 10% HY TME | Extranasal | Sub- | 8 WPI | ND | 8/8 |
| | brain homogenate | | clinical* | | | |
| 35 | 10 μl 10% HY TME | Extranasal | Sub- | 8 WPI | ND | 8/8 |
| | brain homogenate | | clinical* | | | |
| 36 | 10 μl 10% HY TME | Extranasal | Sub- | 8 WPI | ND | 5/8 |
| | brain homogenate | | clinical* | | | |
| 37 | 10 μl 10% HY TME | Extranasal | Sub- | 10 WPI | ND | 8/8 |
| | brain homogenate | | clinical* | | | |
| 38 | 10 μl 10% HY TME | Extranasal | Sub- | 10 WPI | ND | 5/8 |
| | brain homogenate | | clinical* | | | |
| 39 | 10 μl 10% HY TME | Extranasal | Sub- | 10 WPI | ND | 8/8 |
| | brain homogenate | | clinical* | | | |
| 40 | 10 μl 10% HY TME | Extranasal | Sub- | 12 WPI | − | 8/8 |
| | brain homogenate | | clinical | | | |
| 41 | 10 μl 10% HY TME | Extranasal | Sub- | 12 WPI | − | 8/8 |
| | brain homogenate | | clinical | | | |
| 42 | 10 μl 10% HY TME | Extranasal | Sub- | 12 WPI | − | 8/8 |
| | brain homogenate | | clinical | | | |
| 43 | 10 μl 10% HY TME | Extranasal | Sub- | 14 WPI | − | 8/8 |
| | brain homogenate | | clinical | | | |
| 44 | 10 μl 10% HY TME | Extranasal | Sub- | 14 WPI | + | 8/8 |
| | brain homogenate | | clinical | | | |
| 45 | 10 μl 10% HY TME | Extranasal | Sub- | 14 WPI | + | 8/8 |
| | brain homogenate | | clinical | | | |
| 46 | 10 μl 10% HY TME | Extranasal | Sub- | 16 WPI | + | 8/8 |
| | brain homogenate | | clinical | | | |
| 47 | 10 μl 10% HY TME | Extranasal | Sub- | 16 WPI | + | 7/8 |
| | brain homogenate | | clinical | | | |
| 48 | 10 μl 10% HY TME | Extranasal | Sub- | 16 WPI | + | 8/8 |
| | brain homogenate | | clinical | | | |
| 49 | 10 μl 10% HY TME | Extranasal | Sub- | 18 WPI | ND | 8/8 |
| | brain homogenate | | clinical | | | |
| 50 | 10 μl 10% HY TME | Extranasal | Sub- | 18 WPI | ND | 7/8 |
| | brain homogenate | | clinical | | | |
| 51 | 10 μl 10% HY TME | Extranasal | Sub- | 18 WPI | ND | 8/8 |
| | brain homogenate | | clinical | | | |
| 52 | 10 μl 10% HY TME | Extranasal | Clinical | 20 WPI | ND | 8/8 |
| | brain homogenate | | | | | |
| 53 | 10 μl 10% HY TME | Extranasal | Clinical | 20 WPI | ND | 7/8 |
| | brain homogenate | | | | | |
| 54 | 10 μl 10% HY TME | Extranasal | Clinical | 20 WPI | ND | 8/8 |
| | brain homogenate | | | | | |
| 55 | 10 μl 10% sham | Extranasal | NA | 8 WPI | − | 0/8 |
| | homogenate | | | | | |
| 56 | 10 μl 10% sham | Extranasal | NA | 10 WPI | − | 0/8 |
| 57 | homogenate | Extranasal | NA | 12 WPI | − | 0/8 |
| 58 | 10 μl 10% sham | Extranasal | NA | 14 WPI | − | 0/8 |
| 59 | homogenate | Extranasal | NA | 16 WPI | − | 0/8 |
| 60 | 10 μl 10% sham | Extranasal | NA | 18 WPI | − | 0/8 |
| 61 | homogenate | Extranasal | NA | 20 WPI | − | 0/8 |

WPI = weeks post inoculation
NA = Not available
ND = Not done
− = PrP^{D} was not detected
+ = PrP^{D} was detected in the sample
*= Less than/equal to the halfway point to clinical disease

Utilization of hamster models for the propagation and detection of hematogenous PrPD have been used extensively [Saa, P., J. Castilla, and C. Soto, Presymptomatic detection of prions in blood. Science, 2006. 313(5783): p. 92-4; Castilla, J., P. Saa, and C. Soto, Detection of prions in blood. Nat Med, 2005. 11(9): p. 982-5; Orrú, C. D., et al., Prion Disease Blood Test Using Immunoprecipitation and Improved Quaking-Induced Conversion. mBio, 2011. 2(3): p. e00078-11; Hofmann, J. P., et al., Cell-to-cell propagation of infectious cytosolic protein aggregates. Proc Natl Acad Sci USA, 2013. 110(15): p. 5951-6. Murayama, Y., et al., Urinary excretion and blood level of prions in scrapie-infected hamsters. J Gen Virol, 2007. 88(Pt 10): p. 2890-8; Holada, K., et al., Scrapie Infectivity in Hamster Blood Is Not Associated with Platelets. Journal of Virology, 2002. 76(9): p. 4649-4650.], primarily with scrapie infections. Previous to this study, RT-QuIC had not been used to probe for PrP^{C} converting activity in whole blood of TME infected hamsters. To ensure that the detection of RT-QuIC blood-borne PrP^{D} detection was not exclusive to CWD, we analyzed whole blood harvested from IHC-confirmed TME-infected and mock-infected hamsters. We have demonstrated PrP^{C} converting activity in sub-clinical TME infected hamsters with 94.6% sensitivity and 100% specificity. We have also shown that the WBO RT-QuIC assay possesses the ability to detect PrP^{D} in the blood of TME-infected hamsters prior to the mid point between inoculation and clinical disease.

These observations reveal that RT-QuIC is consistently more sensitive in detection of hematogenous PrP^{D} in sub-clinical animals than previously reported for PMCA [Saa, P., J. Castilla, and C. Soto, Presymptomatic detection of prions in blood. Science, 2006. 313(5783): p. 92-4.]. Thus, the WBO RT-QuIC assay may be applicable for the detection of prionemia in multiple species (animals/humans).

### Example 7

### Immunohistochemistry Confirmation of RT-QuIC Results

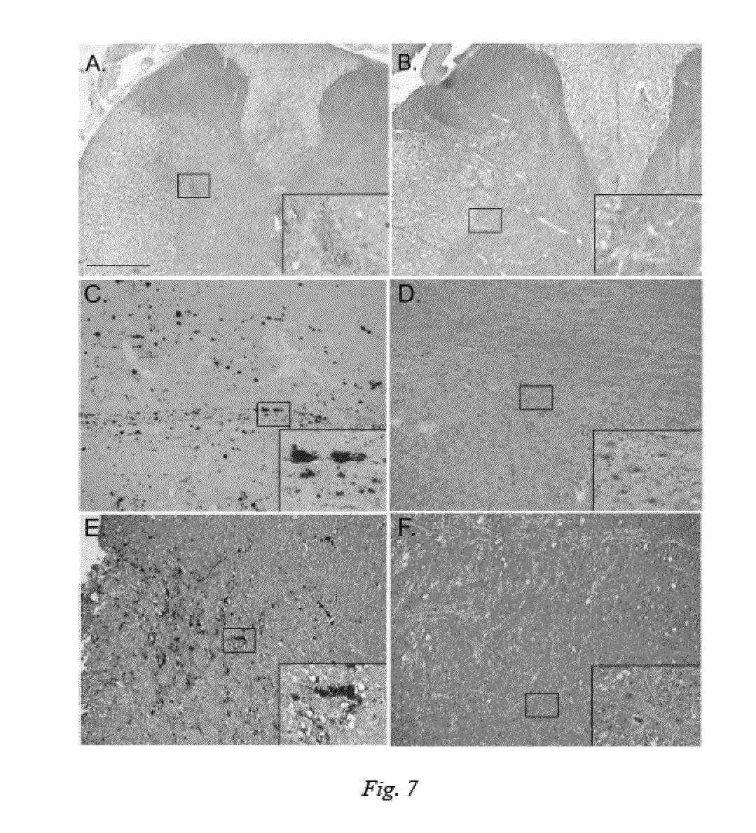

Immunohistochemistry was applied as a confirmation for the presence of PrP^{D} deposition in animals where PrP^{C} converting activity was detected in blood. IHC was performed on both cervid and hamster TME-inoculated and mock-inoculated brains for detection of the disease associated isoform of the prion protein, PrP^{D}. PrP^{D} deposition was observed in TSE-infected animals, but not in mock-inoculated animals (FIG. 7; Tables 1, 2).

### Example 8

### Mouse and Hamster Bioassay Sensitivity Vs. RT-QuIC Sensitivity

To determine the brain equivalent sensitivity of RT-QuIC for TME and CWD samples, RT-QuIC analysis of serial dilutions of TSE-positive brain homogenates were compared to lethal dose bioassay titrations in HY TME-infected hamsters and CWD-infected mice.

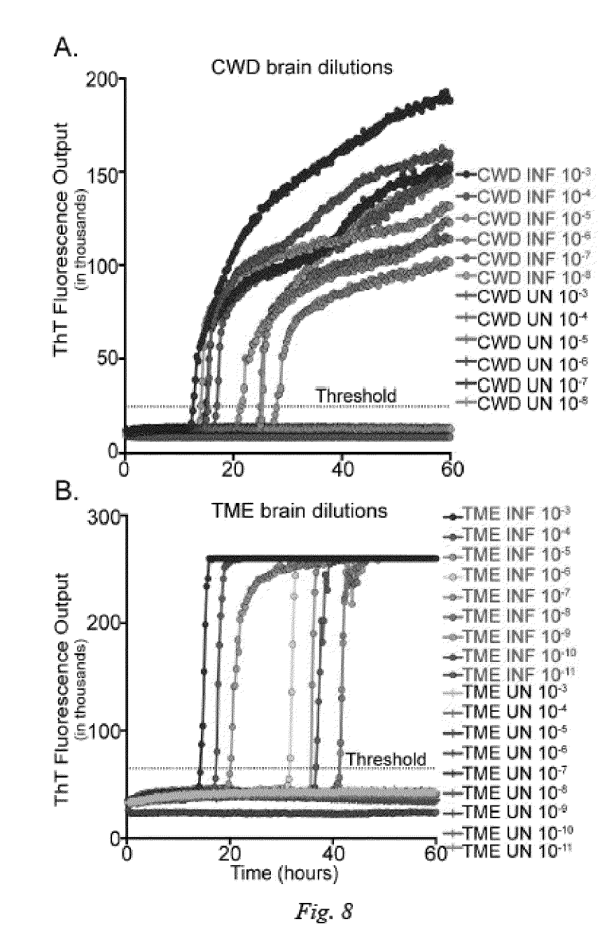

Using bioassay in cervidized transgenic mice, the LD₅₀ titer for CWD-positive brain was determined to be a 0.001%, or 10⁻⁴, brain homogenate (Table 3). End point dilution analysis revealed a failure to cause disease in dilutions greater than 10⁻⁵. Serial dilutions of CWD-positive brain homogenates in RT-QuIC demonstrated consistent positivity to a dilution of 10⁻⁶ (FIG. 8) indicating that the sensitivity of RT-QuIC for CWD detection is equal to or greater than animal bioassay.

TABLE 3
Bioassay of CWD-positive cervid brain in TgCerPrP mice

| Dose (% brain | # Clinical/ | Days post-inoculation (DPI) |
|---|---|---|
| homogentate) | total n | to clinical disease |
| 10 | 7/9^{A} | 137 ± 63 DPI |
| 1 | 9/9 | 200 ± 29 DPI |
| 0.1 | 8/9^{B} | 220 ± 70 DPI |
| 0.01 | 8/9^{B} | 250 ± 68 DPI |
| 0.001 | 7/9 | 397 ± 152 DPI |
| 0.0001 | 1/9 | 335 DPI |
| 0.00001 | 0/9 | NA |

DPI = Days post inoculation
^{A}= 2/9 mice died for reasons unrelated to CWD infection
^{B}= 1/9 mice died for reasons unrelated to CWD infection
NA = Not applicable

The LD₅₀ for hamsters intracranialy inoculated with HY TME was determined to be 10⁻⁸, as demonstrated previously by Kincaid, et al. [Kincaid, A. E. and J. C. Bartz, The nasal cavity is a route for prion infection in hamsters. J Virol, 2007. 81(9): p. 4482-91.]. Endpoint dilution analysis resulted in failure to cause disease in dilutions greater than 10⁻⁹. RT-QuIC analysis of HY TME brain homogenates revealed PrP^{C} converting activity to 10⁻¹⁰ (FIG. 8) indicating that the sensitivity of RT-QuIC for HY TME detection is equal to or greater than animal bioassay.

### Example 9

### Materials and Methods

All animals were handled in strict accordance with guidelines for animal care and use provided by the United States Department of Agriculture (USDA), National Institutes of Health (NIH) and the Association for Assessment and Accreditation of Laboratory Animal Care International (AAALAC), and all animal work was approved by Colorado State University Institutional Animal Care and Use Committee (IACUC) Institutional Animal Care and Use Committee (IACUC) (approval numbers 02-151A, 08-175A and 11-2615A). All procedures involving hamsters were preapproved by the Creighton University Institutional Animal Care and Use Committee and were in compliance with the Guide for the Care and Use of Laboratory Animals.

### Cervid Inoculation

Cervid whole blood was procured from historical and contemporary white-tailed and muntjac deer studies conducted at CSU (Table 1). In brief, naïve 1-2 year old white-tailed deer (Odocoileus virginianus) were inoculated with CWD-positive material as follows: 1) 1.0 g of brain in a 10% brain homogenate administered intracranialy [Mathiason, C. K., et al., Infectious Prions in the Saliva and Blood of Deer with Chronic Wasting Disease. Science, 2006. 314: p. 133-136.]; 2) 250 ml fresh/frozen whole blood administered intravenously/intraperitonealy, respectively [Mathiason, C. K., et al., Infectious Prions in the Saliva and Blood of Deer with Chronic Wasting Disease. Science, 2006. 314: p. 133-136.]; 3) 1.0 g of brain in a 10% brain homogenate administered orally; or 4) 2 ml of a 5% (wt/vol) brain homogenate aerosol-administered [Denkers, N. D., et al., Aerosol transmission of chronic wasting disease in white-tailed deer. J Virol, 2013. 87(3): p. 1890-2.]. Negative control white-tailed deer were exposed to sham inoculum as described above. Naïve 1-2 year old muntjac deer (Muntiacus reevesi) were inoculated with 1.0 g total brain in a 10% brain homogenate administered orally/subcutaneously (Nails et al, PLoS One accepted; publication date pending). Negative control muntjac deer received sham inoculum as described above.

### Hamster Inoculation

Male 10-11 week old Syrian hamsters (Harlan Sprague Dawley, Indianapolis, Ind.) were used in these studies. Extranasal (e.n.) inoculations using a 10% w/v brain homogenate containing 10^{6.8} intracerebral 50% lethal doses per ml of the HY TME agent or a sham homogenate were performed as previously described [Kincaid, A. E., et al., Rapid transepithelial transport of prions following inhalation. J Virol, 2012. 86(23): p. 12731-40.]. Hamsters receiving e.n. inoculations were briefly anesthetized with isoflurane (Webster Veterinary), placed in a supine position and 5 μl of brain homogenate was placed just inferior to each nostril (10 μl total volume). Brain homogenate was immediately inhaled into the nasal cavity, as hamsters are obligate nose breathers.

Blood and Tissue Collection from Cervids

Whole blood (10 ml/cervid/anticoagulant) was collected from n=22 CWD-inoculated cervids—six in various stages of disease presentation and 16 at termination—and from 11 negative control sham-inoculated cervids (Table 1). All blood samples were preserved in one of three anticoagulants: 1) 14% anticoagulant citrate phosphate dextrose adenine (CPDA), 2) 15% ethylenediaminetetraacetic acid (EDTA), or 3) 200 units/ml heparin, before being placed in 1 ml aliquots and frozen at −80° C. Brain (medulla oblongata) collected from each terminal white tailed deer and muntjac deer was frozen at −80° C. or fixed in 10% neutral buffered formalin or paraformaldehyde-lysine-periodate (PLP) and stored in 60% ethanol prior to processing.

Blood and Tissue Collection from Hamsters

At selected time points post-infection, three infected and one mock-infected hamster were anesthetized with isoflurane and blood was collected via cardiac puncture into heparinized tubes for preservation at −80° C. (Table 2). The animals were then transcardially perfused with 50 ml of 0.01 M Dulbecco's phosphate buffered saline followed by 75 ml of McLean's PLP fixative. Brain and brainstem were immediately removed and placed in PLP for 5-7 hours at room temperature prior to paraffin processing and embedding.

### Brain Tissue Homogenization

Ten percent (10%) brain tissue homogenates were created from the obex region of the medulla oblongata by homogenizing 0.5 g brain tissue in 0.5 ml homogenate buffer (1×PBS+0.1% Triton-X 100 [Sigma-Aldrich]). Samples were homogenized using 0.5 mm diameter zirconium oxide beads and a Bullet Blender (Next Advance) for 5 minutes at a speed setting of 10. Homogenates were stored at −80° C. in 20 μl aliquots.

### Whole Blood Freeze-Thaw and Homogenization Process

One milliliter (1 ml) aliquots of whole blood were frozen at −80° C. for 30 minutes and subsequently thawed at 22° C. for 60 minutes. This process was repeated four times. Samples were then homogenized using 0.5 mm diameter zirconium oxide beads and a Bullet Blender (Next Advance) for 5 minutes at top speed.

### Sodium Phosphotungstic Acid (NaPTA) Precipitation

Sodium phosphotungstic acid (NaPTA) precipitation of prions, as first described by Wadsworth et al. [D'Castro, L., et al., Isolation of Proteinase K-Sensitive Prions Using Pronase E and Phosphotungstic Acid. PLOS ONE, 2010. 5(12): p. e15679.], was used to concentrate proteins (including PrP) present in whole blood samples. Frozen whole blood homogenates were thawed and centrifuged at 2000 rpm for one minute to remove cellular debris. Five hundred microliters (500 μl) of supernatant was mixed with an equal volume of 4% sarkosyl in 1× phosphate buffered saline (PBS) and incubated for 30 minutes at 37° C. with constant agitation. Samples were then adjusted to contain a final concentration of 50 U/ml of benzonase (Sigma-Aldrich) and incubated at 37° C. for another 30 minutes with constant agitation. A solution of 4% (w/v) phosphotungstic acid (Sigma-Aldrich) and 170 mM magnesium chloride, adjusted to pH 7.4 with NaOH, was added to the sample for a final concentration of 0.3% (w/v) NaPTA and agitated at 37° C. for 30 minutes. Samples were then centrifuged for 30 minutes at 14,000 rpm and the pellet was resuspended in 50 μl 0.1% (v/v) sarkosyl.

### Recombinant Protein Preparation

Recombinant protein was expressed and purified as previously described [Orru, C. D., et al., Human variant Creutzfeldt-Jakob disease and sheep scrapie PrP(res) detection using seeded conversion of recombinant prion protein. Protein Eng Des Sel, 2009. 22(8): p. 515-21] (D. Henderson, et al; submitted for publication). Truncated recombinant Syrian hamster PrP (SHrPrP 90-231; received from the Caughey laboratory) expressed by Rosetta strain Escherichia coli was inoculated into 1 liter of LB containing Auto Induction™ supplements (EMD Biosciences). Cultures were allowed to grow overnight until harvest when an OD (600 nm) of ˜3 was reached. Cells were lysed using Bug Buster™ and Lysonase™ (EMD Biosciences). Inclusion bodies (IB) were isolated by centrifugation at 15,000×g and were solubilized in 8M guanidine hydrochloride in Tris-phosphate buffer (100 mM NaPO₄ and 10 mM Tris pH 8.0). The protein solution obtained was bound to Super Flow Ni-NTA resin (Qiagen) pre-equilibrated with denaturing buffer (6.0 M GuHCl Tris-phosphate) at room temperature with agitation for 45 minutes and added to a XK FPLC column (GE). SHrPrP was refolded on the column with refolding Trisphosphate buffer at 0.75 ml/min for 340 ml, then eluted with 0.5 M imidazole Tris-phosphate pH 5.5 at 2.0 ml/min for a total of 100 ml. Eluted fractions were collected and dialyzed in two changes of 4.0 dialysis buffer (20 mM NaPO₄ pH 5.5). Following dialysis, purified protein was adjusted to 0.6 mg/ml, flash frozen in 1 ml aliquots, and stored at −80° C.

### Real-Time Quaking Induced Conversion (RT-QuIC) Assay

Real-time quaking induced conversion (RT-QuIC), first described by Atarashi et al. [Atarashi, R., et al., Ultrasensitive detection of scrapie prion protein using seeded conversion of recombinant prion protein. Nat Methods, 2007. 4(8): p. 645-50], Wilham et al. [Wilham, J. M., et al., Rapid end-point quantitation of prion seeding activity with sensitivity comparable to bioassays. PLoS Pathog, 2010. 6(12): p. e1001217.], and Orru et al. [Orrú, C. D., et al., Prion Disease Blood Test Using Immunoprecipitation and Improved Quaking-Induced Conversion. mBio, 2011. 2(3): p. e00078-11], was used for the conversion of small quantities of prions present in the blood of TSE-infected animals. Positive assay controls and samples consisted of serial dilutions of CWD/TME-infected brain (10⁻³-10⁻⁹) and blood (10⁰-10⁻⁶), respectively. Negative assay controls and samples were comprised of CWD/TME-naïve brain (10⁻³-10⁻⁹) and blood (10⁰-10⁻⁶), respectively. RT-QuIC reactions were set up in 96-well clear bottom optic plates (Nalgene Nunc) and consisted of 98 μl RT-QuIC Buffer (final concentrations of 1×PBS, 1 mM EDTA, 10 μM Thioflavin T (ThT), 100-200 mM NaCl buffer, and 0.1 mg/ml recombinant Syrian hamster PrP^{C} substrate and 2 μl sample. Once reactions were set up in each well, plates were placed in a BMG Fluostar fluorescence plate reader with settings of 42° C. for 60 hours with cycles consisting of 1 minute shake, 1 minute rest and ThT fluorescence measurements were taken every 15 minutes. Data were removed from the fluorometer and processed using Microsoft Excel (Microsoft Inc.) prior to graph production with Prism 6 (GraphPad Prism).

### Cervid Immunohistochemistry

Samples were processed and analyzed as previously described (Nails et al, PLoS One accepted; publication date pending) In brief, fixed tissues were treated with formic acid, embedded in paraffin, cut and placed on positively charged slides. Deparaffinized, rehydrated and PK digested (20 mg/ml) tissues underwent epitope retrieval and were probed with primary antibody BAR224 (Cayman Chemical) and secondary anti-mouse HRP labeled polymer (Dako) prior to counterstain and reading by light microscopy

### Hamster Immunohistochemistry

Immunohistochemistry was performed to detect PrP^{D} as previously described [Kincaid, A. E., et al., Rapid transepithelial transport of prions following inhalation. J Virol, 2012. 86(23): p. 12731-40.]. In brief, deparaffinized, formic acid treated tissue sections were processed for antigen retrieval. Endogenous peroxidase and non-specific staining were blocked in H₂O₂ in methanol and normal horse serum. The sections were probed with monoclonal anti-PrP antibody 3F4 followed by secondary biotinylated horse anti-mouse immunoglobulin G conjugate prior to detection with ABC solution (Elite kit; Vector Laboratories). The sections were counterstained with hematoxylin and read by light microscopy

### Western Blotting

Western blotting performed as previously described [Denkers, N. D., G. C. Telling, and E. A. Hoover, Minor oral lesions facilitate transmission of chronic wasting disease. J Virol, 2011. 85(3): p. 1396-9.62] with the following modifications: tissue homogenates were mixed with proteinase K (PK) (Invitrogen) to a final concentration of 50 μg/ml and incubated at 37° C. for 30 minutes, followed by incubation at 45° C. for 10 minutes with constant agitation. Samples were size fractionated on a NuPAGE 10% Bis-Tris gel (Novex) in 1×MOPS buffer at 100 volts for 2.5 hours, transferred to a polyvinylidene fluoride (PVDF) membrane for 7 minutes using the Trans-blot Turbo transfer system (Biorad). Post-transfer, the PVDF membrane was loaded onto a wetted SNAP i.d. holder (Millipore) and placed in the SNAP i.d. vacuum filtration system (Millipore). The PVDF membrane was blocked for 10 minutes with Blocking Buffer (Blocker casein in TBS [Thermo Scientific] with 0.1% Tween 20), and incubated for 10 minutes with 0.2 μg/ml primary antibody BAR224 (Cayman Chemical)—HRP conjugated antibody. The membrane was washed with TBST and developed using ECL Plus enhanced chemiluminescence Western blotting detection reagents (Invitrogen) and imaged on a Luminescence image analyzer LAS 3000 (Fujifilm).

### Mouse Titration Bioassay

All animals were handled in strict accordance with guidelines for animal care and use provided by the United States Department of Agriculture (USDA), National Institutes of Health (NIH) and the Association for Assessment and Accreditation of Laboratory Animal Care International (AAALAC), and all animal work was approved by Colorado State University Institutional Animal Care and Use Committee (JACUC). Seven cohorts of TgCerPrP mice (n=9) were inoculated with 30 il of a CWD-infected cervid brain homogenate intracranialy. Each cohort received a different concentration of inoculum ranging from 10% (w/v) to 0.00001% (w/v). Negative control mice were inoculated with sham material. Mice were subsequently observed and terminated upon onset of clinical disease. All mice were analyzed for the presence of PrPD by Western blot and immunohistochemistry.

### GLOSSARY OF CLAIM TERMS

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

Other than in the operating examples, or unless otherwise expressly specified, all of the numerical ranges, amounts, values and percentages such as those for amounts of materials, times and temperatures of reaction, ratios of amounts, values for molecular weight (whether number average molecular weight (“Mₙ”) or weight average molecular weight (“M_{w}”), and others in the following portion of the specification may be read as if prefaced by the word “about” even though the term “about” may not expressly appear with the value, amount or range. Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used.

As used herein, the term “comprising” is intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

All references cited in the present application are incorporated in their entirety herein by reference to the extent not inconsistent herewith.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A method of screening a blood sample for the presence of prions comprising the steps of:
collecting the blood sample in heparin;
contacting the sample with a solution comprising recombinant prion protein (rPrP) and Thioflavin T (ThT); and
measuring the resulting ThT fluorescence in the sample.

2. The method according to claim 1 wherein about 200 units/ml of heparin are added to the sample.

3. The method according to claim 1 further comprising the step of freezing and thawing the sample prior to contacting the sample with a solution comprising recombinant prion protein (rPrP) and Thioflavin T (ThT).

4. The method according to claim 3 wherein aliquots of whole blood were frozen at about −80° C. for about 30 minutes and subsequently thawed at about 22° C. for about 60 minutes.

5. The method according to claim 3 wherein the samples are subjected to at least four (4) freeze-thaw cycles.

6. The method according to claim 3 further comprising the step of homogenizing the sample after the freezing and thawing steps.

7. The method according to claim 1 further comprising the step of precipitating the prions in sodium phosphotungstic acid (NaPTA) prior to contacting the sample with a solution comprising recombinant prion protein (rPrP) and Thioflavin T (ThT).
